(19) United States
(12) Patent Application Publication
(10) Pub. No.: US 2025/0359799 A1
(43) Pub. Date: Nov. 27, 2025

(54) ELECTROMYOGRAPHY DEVICES AND METHODS WITH FILTERING OF ELECTROMYOGRAPHY SIGNALS

(71) Applicant: Battelle Memorial Institute, Columbus, OH (US)

(72) Inventors: Nicholas J. Tacca, Columbus, OH (US); Eric C. Meyers, Columbus, OH (US); Ian W. Baumgart, Albany, OH (US)

(21) Appl. No.: 19/215,461

(22) Filed: May 22, 2025

Related U.S. Application Data
(60) Provisional application No. 63/650,461, filed on May 22, 2024.

(51) Int. Cl. A61B 5/296; A61B 5/00; A61B 5/256; A61N 1/36

(52) U.S. Cl. CPC A61B 5/296; A61B 5/256; A61B 5/7207; A61N 1/36003; A61N 1/36031

(57) ABSTRACT

An electromyography (EMG) measurement system includes a garment configured to be worn on an anatomical region, electrodes arranged on the garment to contact skin of the anatomical region when the garment is worn on the anatomical region, electronics connected with the electrodes to measure EMG data emanating from the anatomical region, and an electronic processor programmed to filter the EMG data to suppress or remove artifacts using filters computed using approximate joint diagonalization of covariance (AJDC) matrices or by transforming the EMG data to source signals using iteratively adjusted forward filters.

This application claims the benefit of U.S. provisional application Ser. No. 63/650,461 filed May 22, 2024, which is incorporated herein by reference in its entirety.

### BACKGROUND

The following relates to the electromyography arts, neuromuscular electrical stimulation arts, neuromuscular therapy arts, neuromuscular rehabilitation arts, virtual reality arts, augmented reality arts, and to the like.

The following relates to improvements in electromyography (EMG) measurement and analysis, and to applications of same in diverse fields such as: neuromuscular electrical stimulation (NMES) guided by EMG signals; EMG-based assessment of neuromuscular debilitation due to spinal cord injury (SCI), stroke, traumatic brain injury (TBI), or other pathologies such as Parkinson's disease; neuromuscular therapy and/or rehabilitation performed using or guided by EMG measurements; EMG-guided muscle tremors suppression; virtual reality (VR) or augmented reality (AR) systems utilizing EMG measurements to monitor participant activity and/or guide VR or AR content presentation; sports performance or technique analysis; injury recovery; fatigue assessment; and like applications.

In performing such tasks, accurate measurement of EMG signals can be challenging. For example, artifacts can be introduced into the EMG signals by diverse sources such as electrode impedance changes caused by shifting of the electrodes or sweat, radio frequency interference (RFI) from local electronics, and so forth. If EMG measurements are interleaved with NMES pulses, for example in EMG-guided NMES applications, then the NMES pulses can also interfere with the EMG signal measurement.

Various improvements are disclosed herein.

### BRIEF SUMMARY

In accordance with some illustrative embodiments disclosed herein, an electromyography (EMG) measurement device comprises: a garment configured to be worn on an anatomical region of an associated wearer; a plurality of electrodes arranged on the garment to contact skin of the anatomical region when the garment is worn on the anatomical region of the associated wearer; electronics operatively connected with the plurality of electrodes and configured to measure EMG data emanating from the anatomical region; and an electronic processor programmed to filter the EMG data to suppress or remove artifacts using filters computed using approximate joint diagonalization of covariance (AJDC) matrices or by transforming the EMG data to source signals using iteratively adjusted forward filters.

In accordance with some illustrative embodiments disclosed herein, an EMG measurement method includes: measuring EMG data emanating from an anatomical region; and filtering the EMG data to suppress or remove artifacts using filters computed using approximate joint diagonalization of covariance (AJDC) matrices or by transforming the EMG data to source signals using iteratively adjusted forward filters.

In accordance with some illustrative embodiments disclosed herein, an electronic processor is programmed to perform motor unit action potential (MUAP) extraction on EMG data by operations including: transforming the EMG data to source signals using forward filters; computing power signals corresponding to the source signals by squaring the respective source signals; and identifying the MUAPs from the computed power signals using peak detection.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows an electromyography (EMG) measurement system, together with an optional neuromuscular electrical stimulation (NMES) capability.

FIG. 2 diagrammatically shows a flowchart of a method for fitting approximate joint diagonalization of covariance (AJDC) filters used in the EMG measurement system of FIG. 1 for EMG artifact removal and motor unit action potential (MUAP) extraction.

FIG. 3 diagrammatically shows a flowchart of EMG artifact removal using the AJDC filters fitted by the method of FIG. 2.

FIG. 4 diagrammatically shows a flowchart of a cycle of MUAP extraction using the AJDC filters fitted by the method of FIG. 2 and operating on the filtered EMG data produced by the EMG artifact removal method of FIG. 3.

### DETAILED DESCRIPTION

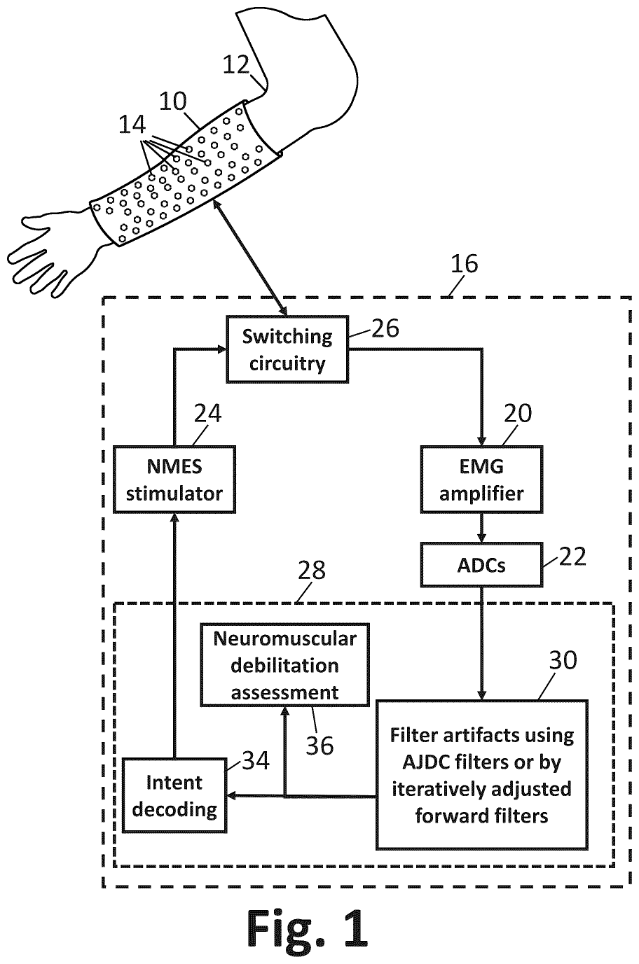

With reference to FIG. 1, an electromyography (EMG) measurement system is shown, which in the illustrative example also includes an optional neuromuscular electrical stimulation (NMES) capability. The EMG measurement system includes a garment 10 that is wearable on an anatomical region 12, and that includes a plurality of electrodes 14 arranged to contact skin of the anatomical region 12 when the garment is worn on the anatomical region. The illustrative garment 10 is a sleeve 10 worn on an arm 12. The garment 10 may be made of a cloth, textile, leather, polyester, or other material, and is sized and shaped to be worn on the anatomical region 12 from which EMG is to be measured. The garment 10 may more generally, for example, be a sleeve that is sized and shaped to be worn on an arm, a leg, a wrist, an ankle, an arm and a wrist, a leg and an ankle, a torso, or so forth. By way of some further examples, suitable garments for a hand would include, for example, a glove or mitten. Suitable garments for a foot would include, for example, a sock or boot. The glove, mitten, sock, or boot can be extended over the wrist or ankle to provide a garment for a wrist and hand or for an ankle and foot, or further extended to provide a garment for an arm and wrist and hand or for a leg and ankle and foot. These are merely non-limiting illustrative examples.

The sizing of the garment 10 is suitably subject-specific to account for different anatomies of different persons; or, the garment 10 may be designed to be adjustable for anatomical differences between persons—for example, the illustrative sleeve 10 could employ a wrap-around arrangement with Velcro that can be adjustably wrapped around arms of different diameters. The plurality of electrodes 14 are disposed on the inside of the garment 10 to contact the skin of the anatomical region 12 when the garment 10 is worn on the anatomical region 12. For example, the electrodes 14 may be embedded on the inside of a stump interface of a prosthetic device, prosthetic liner, or soft exoskeleton/exosuit. Note that FIG. 1 illustrates the garment 10 as transparent to reveal the underlying electrodes 14, but more typically the garment will be translucent or opaque. The electrodes 14 are connected by wires (possibly woven into the garment 10), circuitry of flexible printed circuit boards, and/or so forth to connect with associated electronics 16. The various components of the electronics 16 may be integrated with the garment 10, or separate from the garment 10 and connected with the electrodes 14 by suitable electrical wires or cables or the like. Typically, the electrodes 14 are surface electrodes, i.e., transcutaneous electrodes; however, embodying the electrodes 14 as needle electrodes or the like is also contemplated. In some embodiments, the garment 10 is an elastic garment whose elasticity provides compressive force holding the electrodes 14 firmly against the skin of the wearer. Such garment elasticity can also in some specific implementations facilitate the garment 10 being wearable on arms (or other target anatomical region 12) of different sizes. The electrodes 14 are designed to provide good electrical contact with the skin of the anatomical region 12. For example, the electrodes 14 may be electrogel discs, or may comprise an electrically conductive polymer electrode material such as a mixed ionic-electronic conducting (MIEC) material, or so forth. Optionally, the garment 10 may further include other devices such as one or more inertial measurement unit (IMU) devices (not shown) such as an accelerometer, gyroscope, or the like, to provide information on the spatial orientation of the sleeve 10 (and hence of the anatomical region 12).

The electrodes 14 are used to measure EMG signals produced by the anatomical region 12. The EMG signal measurements are potential difference measurements between pairs of electrodes 14, where the pairs of electrodes are pairs of electrodes of the array or, in a monopolar configuration, each pair is an electrode of the array and a common reference electrode. Each such pair of electrodes is referred to herein as an EMG channel. To this end, the electronics 16 include an EMG amplifier 20, which may for example comprise an operational amplifier (op-amp) based amplifier circuit. It will be appreciated that the EMG amplifier 20 is a multi-channel amplifier, e.g. each EMG channel (corresponding to an electrode 14: reference-electrode pair, or to a pair of electrodes 14) is separately received and amplified in parallel by the multi-channel EMG amplifier 20. Preferably, the outputs of the multichannel EMG amplifier 20 are digitized by analog-to-digital converters (ADCs) 22. By way of nonlimiting illustrative example, the combination of the multichannel EMG amplifier 20 and multichannel ADC 22 can be embodied as an Intan EMG amplifier (available from Intan Technologies, Los Angeles, California, USA).

The measured EMG can be utilized in various ways. For example, if the subject is suffering neuromuscular debilitation due to spinal cord injury (SCI), stroke, traumatic brain injury (TBI), or pathologies such as Parkinson's disease, then the EMG can be used to assess the extent to which the motor cortex of the subject's brain is able to transmit motor control neural signals to muscles of the anatomical region 12, and the accuracy of such neural signal transmission if present (e.g., if the subject's volition is to move the index finger then do the transmitted neural signals reach the muscles that cause movement the of the index finger, or are the neural signals mis-transmitted to different muscles due to the neuromuscular debilitation). As another example, if the EMG measurement system is deployed in a virtual reality (VR) or augmented reality (AR) system, then the measured EMG can be used to monitor participant activity, guide the VR or AR content presentation, or so forth. These are some nonlimiting illustrative examples of uses the measured EMG in various applications.

With continuing reference to FIG. 1, the EMG measurement system optionally further includes NMES capability, that is, the ability to apply neuromuscular electrical stimulation to the anatomical region 12 using the electrodes 14. To this end, the electronics 16 further include an NMES stimulator 24. NMES may be applied for various reasons, such as (but not limited to): providing functional electrical stimulation (FES); suppressing muscular tremors; promoting regeneration of damaged nerves; inducing somatosensation (e.g., the sensation of touch, raindrops, an arachnid crawling across the skin, or so forth); and/or et cetera. The configuration of the applied NMES (e.g., which subset of the electrodes 14 apply the NMES, the magnitude of the applied NMES, which may vary spatially over the skin of the anatomical region 12, and so forth) may optionally be guided by the measured EMG, after suitable analysis of the EMG. For example, an SCI patient may have residual motor neuron connectivity between the motor cortex and the musculature of the target anatomy 12, but this motor neuron connectivity may be insufficient to cause the muscle contraction necessary for volitional control of the anatomical region 12. In such a case, the residual motor neuron connectivity may be detected as measured EMG at the muscles intended to be contracted, and FES can then be applied to cause the muscles to actually contract thereby moving the anatomical region 12 in accordance with the volitional intent of the wearer of the garment 10.

To implement the optional NMES capability, the electronics 16 further includes an NMES stimulator 24. To enable switching between applying NMES using the NMES stimulator 24 and receiving EMG measurements via the EMG amplifier 20, suitable switching circuitry 26 is provided, including solid state relays, high voltage field effect transistor (FET) components, and so forth, to enable the same set of electrodes 14 to switch between applying NMES stimulation and measuring EMG. (It is noted that if the EMG measurement system does not include NMES capability, then both the NMES stimulator 24 and the switching circuitry 26 may be omitted) to perform NMES, the NMES stimulator 24 generates suitable electrical pulses that are applied to the anatomical region 12 (or a selected portion thereof) by a selected subset of the electrodes 14. In some nonlimiting illustrative embodiments, the applied NMES may comprise NMES pulse waveforms including monophasic and/or biphasic pulses with a voltage between 80 to 300 Volts inclusive or higher. In one specific example, the NMES pulse waveform is a monophasic pulse with a peak current of 0-20 mA which is modulated to vary strength of muscle contraction, frequency of 50 Hz, and a pulse width duration of 500 ms. Again, these are non-limiting illustrative examples. Analogously to the EMG amplifier 20, it will be appreciated that the NMES stimulator 24 is a multichannel NMES stimulator that can in general independently apply different NMES to different channels (where a channel corresponds to an electrode 14: reference-electrode pair, or to a pair of electrodes 14).

Extraction of useful information from the digitized EMG data output by the amplifier 20 and ADCs 22 is challenging. The EMG signal is typically weak, and numerous artifact sources may be present, such as electrode impedance changes caused by shifting of the electrodes 14 or sweat, radio frequency interference (RFI) from local electronics, and so forth. If the EMG measurements are interleaved with NMES pulses applied by the NMES stimulator 24, for example in EMG-guided NMES applications, then the NMES pulses can also interfere with the EMG signal measurement.

To isolate and suppress or remove artifacts from the EMG data, the electronics 16 may further include a computer, microprocessor, or other electronic processor 28 that is programmed to perform processing 30 of the EMG signal to suppress or remove artifacts using filters computed using approximate joint diagonalization of covariance (AJDC) matrices. These filters produced using AJDC matrices are also referred to herein as AJDC filters, and as further described later herein include forward and backward filters and associated thresholds for identifying artifact sources. The illustrative AJDC filters used herein are an example of a second order blind source separation (BSS) method. While AJDC filters are described herein for performing the filtering 30 of the EMG data, other types of BSS methods, such as independent component analysis (ICA), are contemplated for performing the EMG filtering. In another approach, the processing 30 of the EMG signal suppresses or removes artifacts or by transforming the EMG data to source signals using iteratively adjusted forward filters.

The filtering 30 in some embodiments may include extracting motor unit action potentials (MUAPs). The filtering processing 30 can be done in real-time to provide filtered EMG data and/or MUAP activity in real time.

In the illustrative example of FIG. 1, two nonlimiting examples of applications utilizing the MUAP activity are shown. In one example, the electronic processor 28 is further programmed to perform intent decoding processing 34 to determine volitional intent of the user (i.e., the person wearing the garment 10). In this example application, the user may be an SCI patient who has residual motor neuron connectivity between the user's motor cortex and the user's musculature of the target anatomy 12, but this residual motor neuron connectivity is insufficient to cause the muscle contraction necessary for volitional control of the anatomical region 12. The intent decoding 34 determines the user's volitional intent by detecting which motor units are receiving the residual motor neuron signals (as manifested by MUAP activity of those muscle units and/or associated motor neurons obtained by the processing 30), and the NMES stimulator 24 then applies functional electrical stimulation to the identified muscles via the electrodes 14 to cause the anatomical region 12 to perform the movement volitionally intended by the user. In this application, it will be appreciated that the artifact removal processing 30 advantageously removes artifacts produced by the NMES applied during stimulation time intervals that are interleaved with EMG measurement intervals during which the EMG is measured and processed (including the filtering 30 with MUAP activity extraction and intent decoding 34).

In another illustrative application utilizing the MUAP activity, the electronic processor 28 is further programmed to perform neuromuscular debilitation assessment 36. This can take various forms. For example, in one approach the user (who is suffering from neuromuscular debilitation due to SCI, stroke, TBI, or another pathology such as Parkinson's disease) is asked to perform a movement of the anatomical region 12. The user makes the effort but is unable to perform the movement, or performs the movement poorly. The MUAP activity obtained by the processing 30 during this effort is processed by the assessment processing 36 to determine the strength of motor neural signals delivered to the anatomical region 12 during the user's effort, as well as information on how accurately those motor neural signals are targeted to the correct (versus incorrect) muscles.

The applications 34 and 36 diagrammatically shown in FIG. 1 are nonlimiting examples. More generally, the filtered EMG data produced by the filtering 30 can be used in various applications such as: EMG-guided NMES (i.e., using volitional intent obtained from the measured EMG via intent decoding 34); EMG-based assessment 36 of neuromuscular debilitation due to SCI, stroke, TBI, Parkinson's disease, or so forth; neuromuscular therapy and/or rehabilitation performed using or guided by EMG measurements; EMG-guided muscle tremors suppression; VR or AR systems utilizing EMG measurements to monitor participant activity and/or guide VR or AR content presentation; and like applications.

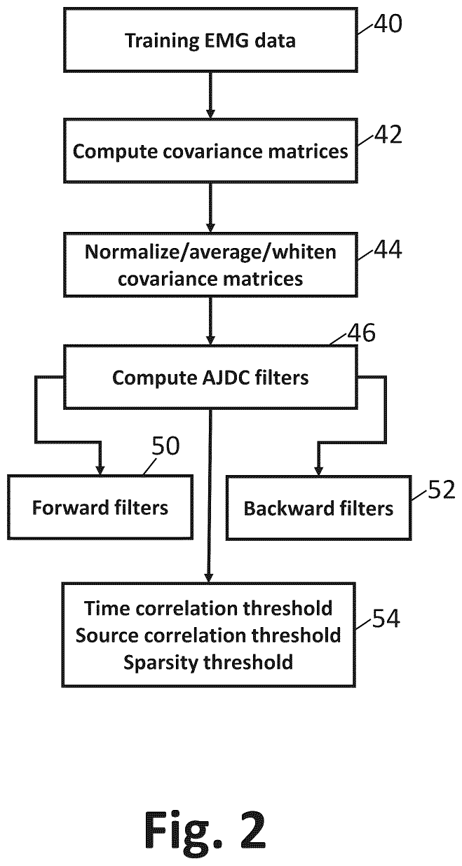

With reference now to FIG. 2, in embodiments in which the EMG filtering 30 of FIG. 1 uses AJDC filters, the AJDC filters used in the EMG filtering 30 are fitted using a method shown in FIG. 2.

Sampled data from training EMG data 40 (or the full training EMG dataset 40) containing both clean training EMG data and training EMG data with artifacts is used for fitting the filter. Optionally, the training EMG data 40 can be extended in time such that separated sources map to current and delayed observations by channel. The training EMG data 40 may be within subject (i.e., all the training EMG data 40 may be measured for a single subject, either in a single session or collected over multiple sessions) to fit AJDC filters tailored for that subject; or, the training EMG data 40 may be acquired for multiple subjects, and/or over multiple sessions, to fit AJDC filters that are generalized for the cohort of subjects represented by the training EMG data 40.

In an operation 42, covariance matrices are computed from the input training EMG data 40 to generate a smooth differentiable manifold on which the original data lies (n_domains×n_samples×n_features×n_features). In one suitable approach, the operation 42 calculates Fourier cospectral covariance matrices to enhance separation of sources in the spectral domain. Alternative methods for the operation 42 include the empirical covariance with/without regularization, or feature-wise kernel methods (such as the Laplacian, sigmoidal, or cosine kernels, or so forth). In an optional operation 44, the covariance matrices are normalized and averaged across domains (in implementations in which the training EMG dataset 40 is acquired across multiple subjects and/or sessions). The subsequent covariance matrices are whitened to center the data (with or without a dimension reduction).

In an operation 46, AJDC filters are computed. In one suitable approach, diagonal filters are approximated using an approximate joint diagonalization algorithm. In one suitable computation approach, Quasi-Newton joint approximation is used to increase speed and separability of sources. Both forward filters 50 and backward filters 52 are computed in the operation 46 from the approximated diagonal filters and whitening filters to transform to the source space (using the forward filters 50) and from source space back to the time domain (using the backward filters 52).

Additionally, the operation 46 determines one or more thresholds 54 for detecting artifacts in the EMG data. When fitting, the filter automatically detects outlier sources (which are expected to be artifacts) based on preset thresholds of what constitutes an outlier source. Kurtosis, time-correlation, median absolute deviation (MAD), sparsity, and source correlation are some suitable methods for automatically distinguishing artifact sources from true signals. In some examples, a time-correlation threshold can be fitted to capture sources with repeating artifacts (e.g. in the case of a repeated artifact from NMES pulses produced by the NMES stimulator 24 if the training EMG data 40 includes training EMG data collected during interleaved EMG measurement/NMES stimulation intervals). A source correlation threshold can be fitted to detect sources that are highly correlated, which can help find artifacts from electrical stimulation contained in multiple sources. A sparsity threshold can be fitted to detect localized artifact sources. The fitting of these threshold can be done using the training EMG data 40 with the subject artifact sources labeled, and the thresholds are optimized to optimally distinguish the labeled artifacts from the remainder of the training EMG data.

BSS inverse/backward filters can advantageously elucidate where the artifact source is localized in reference to the original EMG data. In the case of FES, the artifact originates from the electrodes that produce electrical stimulation. In comparison to the rest of an HD-array of electrodes, this is localized in a small sparse region of the array. Therefore, a sparsity threshold can be used to determine which backward filters 52 are sparse, indicating a likely source of artifact, which can be suppressed. Since the backward filters 52 originate at the stimulation patterns, it is likely that this entire source selectively encodes stimulation artifact and can thus be removed. This should not remove any of the true EMG signal from the associated EMG channels. It merely suppresses the artifact from the original EMG data by suppressing the artifact source associated that is only active during artifact periods.

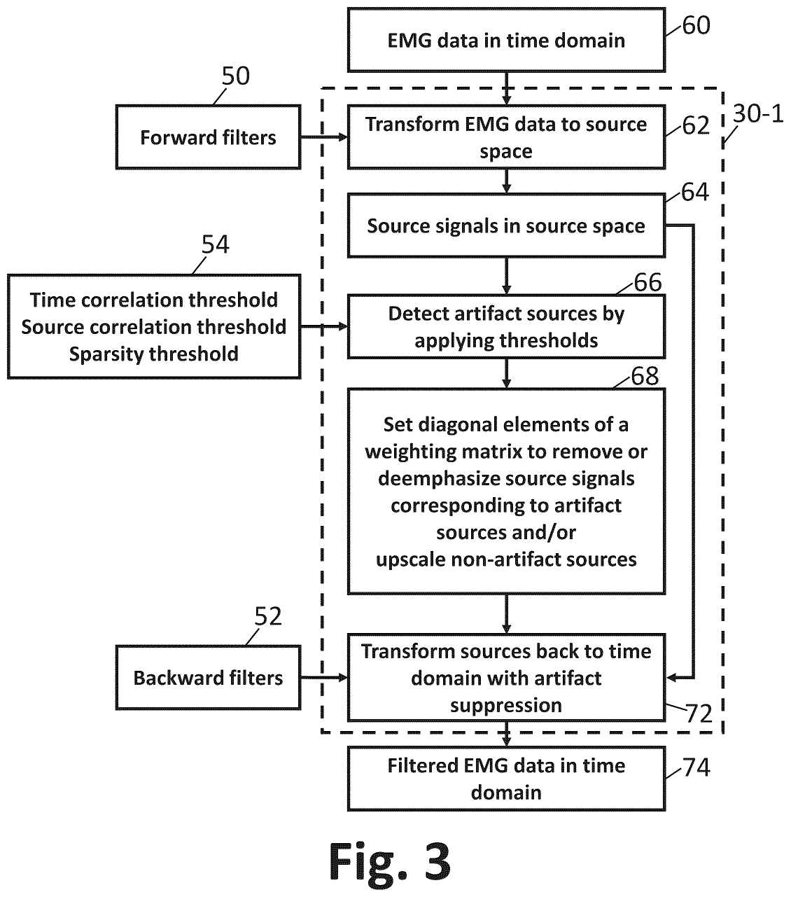

With reference now to FIG. 3, an illustrative example of an embodiment of the EMG filtering process 30 of FIG. 1 is shown, which uses using AJDC matrices. In FIG. 3, the illustrated embodiment of the EMG filtering 30 is denoted as EMG filtering 30-1. EMG data 60 in the time domain is transformed in an operation 62, which uses the fitted forward filters 50 to perform the transformation, thereby producing source signals in source space 64. In an operation 66, artifact sources are detected in the source signals 64 in source space using the fitted thresholds 54. The time-correlation threshold is applied to identify sources with repeating artifacts, such as from repetitive NMES pulses. The source correlation threshold is applied to identify sources that are highly correlated, which can help find artifacts from electrical stimulation contained in multiple sources. The sparsity threshold is applied to detect localized artifact sources.

To suppress or remove the artifact sources detected by the operation 66, an operation 68 sets diagonal elements of a weighting matrix corresponding to artifact sources to zero, and/or upscales diagonal elements of the weighting matrix not corresponding to artifact sources. The approach of upscaling diagonal elements of the weighting matrix not corresponding to artifact sources amplifies good sources (i.e., sources without artifacts exceeding one or more of the thresholds 54) by increasing the weighting of the sources when transforming back into the original data space. An example implementation entails calculating the fisher score or mutual information to determine discriminability of sources in reference to training labels. The ranking of sources by information can be used to weight the sources along the diagonals of the weighting matrix. It is also contemplated to directly calculate features and decode from source space if desired.

In one approach, in the operation 68 artifact sources of an identity matrix are set to zero. This is multiplied with the backward filters 52 which is multiplied with the source signal in an operation 72 to return back to the original signal. The identity matrix determined in the operation 68 acts as a scaling factor or source weighting. In one approach for constructing the weighting matrix, starting with an identity matrix, diagonal elements of the identity matrix are changed to suppress artifact sources completely (by setting the diagonal elements corresponding to the artifact sources to zero), or diagonal elements corresponding to artifact sources can be set to some other weighting value to suppress or deemphasize artifact sources. Additionally or alternatively, diagonal elements of the identity matrix can be upscaled for sources not corresponding to artifact sources to upscale the non-artifact sources (or, additionally or alternatively to upscale the most informative sources). The output of the transformation 74 is filtered EMG data in the time domain, with artifact sources deemphasized or removed and/or non-artifact (or most informative) sources enhanced relative to the artifact sources.

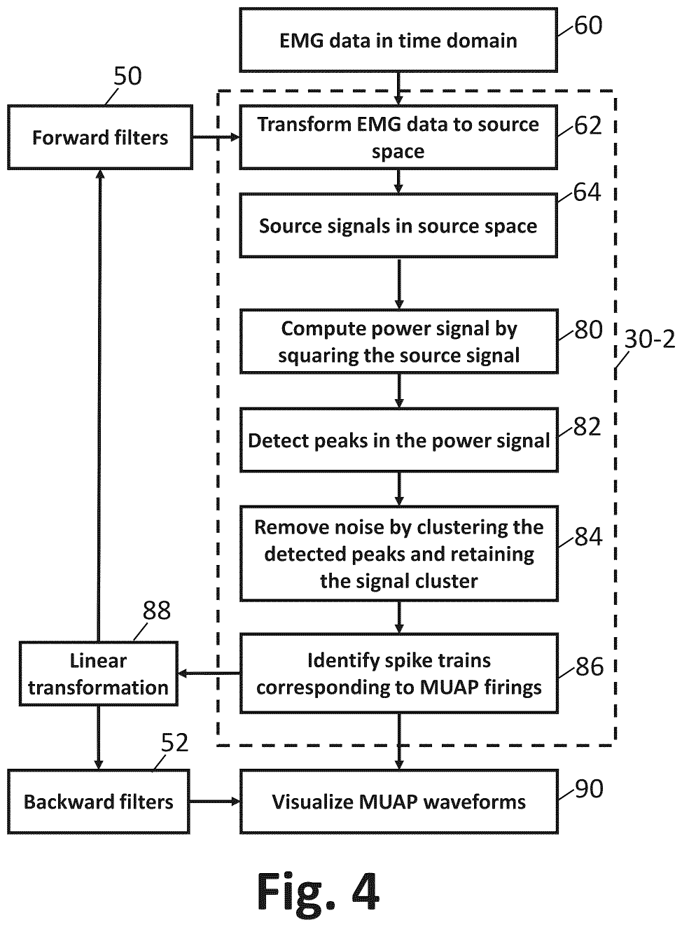

With reference now to FIG. 4, an illustrative example of a cycle according to another embodiment of the processing 30 of FIG. 1 (denoted in FIG. 4 as processing 30-2) is shown. The illustrative MUAP extraction process 30-2 of FIG. 4 operates in source space; accordingly, it receives as input the source signals in source space 64 computed by transform 62 using forward filters 50 as previously described with reference to FIG. 3. To perform the MUAP extraction 30-2, in an operation 80 the signal power is computed by squaring the source signal. In an operation 82, peaks are detected in the power signal based on optional height and inter-spike interval requirements. The squared source signal can be visualized as a signal that is close to zero for most of the time, and then there are large intermittent spikes. The spikes are detected via a peak detection method. It is noted the operations 80 and 82 are performed for each source signal; that is, power signals corresponding to source signals are computed by squaring the respective source signals. In an operation 84, peak signals are clustered using k-means (or another suitable clustering algorithm) into two groups to distinguish signal from noise, and peak indices within the signal cluster are kept and the detected noise peak indices are discarded.

FIG. 4 illustrates one cycle of the MUAP extraction process 30-2, which is performed iteratively to add sources (concatenate with forward/backward filters 50, 52). Criteria to accept new sources may, for example, be based on a similarity metric with sources that have been already found, such as cosine similarity, and/or based on the pulse-to-noise ratio of the new sources based on a threshold (i.e., removing low pulse-to-noise ratio signals). New sources satisfying the criteria are added to the forward/backward filters, for example implemented as a linear transform 88 that is applied to the forward filters 50 to be used in the next iteration sampling the original EMG data 60. This enables iteratively finding new sources in real time. The disclosed method advantageously can identify multiple sources at a time. In this iterative/cyclical process, BSS filters (e.g., implemented as the illustrative linear transformation 88) can be computed to transform the original signal to source. The filters are compared to previous filters already calculated and only new sources are kept. Additionally or alternatively, the resulting source signal/and or spike train can be analyzed to determine whether a source is an artifact. For example, if the pulse-to-noise ratio is low, this may indicate this is not a clean motor unit (MU) source and should not be retained. To retain the new sources, the new forward/backward filters 50 and 52 (as modified by the linear transformation 88) are concatenated with the previous forward/backward filters and continue the iterative loop is continued by sampling the original data and repeating the process. This continues until no new sources are identified. Additionally or alternatively, a limit on the number of cycles may be set (e.g. only go through the loop five times or only until 120 sources are found, for example).

In the illustrative example, the linear transformation 88 representing weights of identified spike trains 86 corresponding to MUAP firings is applied using the forward filters 50 to transform the original time domain signal 60 into the source domain. The forward filters 50 modified by the weightings implemented as the linear transformation 88 selectively weight the original EMG channels such that individual sources are separated. So, for example, if the input EMG data 60 comprises n_samples×n_channels, then that signal is transformed into sources (n_samples×n_sources). The number of sources can be less than, equal to, or greater than the number of channels. The unique number of sources that are now separated (source signals 64) from the original mixed data signal are thus obtained. This can be viewed as unmixing a mixed signal 60 that is recorded into its unique sources 86. The sources 86 in the example of FIG. 4 are suitably motor units that descend from the central nervous system.

In the operation 86, spike trains corresponding to MUAPs are identified. In one approach, binary spike trains or sample arrays are identified based on peak index, and are then returned in a suitable format such as binned (e.g. one spike per MU per bin) or within bins in spikes per bin samples. From there, the cumulative spike train (CST) can be determined across all motor unit sources or for motor units that fire selectively during different movements. The smoothing of the CST is referred to as neural drive, which can be used to train decoders. Alternatively, other embodiments find the power spectrum of motor unit firing (e.g. in the beta band) to indirectly obtain motor cortex commands from EMG data. Optionally, average coherence of randomly sampled CSTs can additionally provide information about the input command from motor cortex as well.

The output of the operation 86 is the extracted MUAPs represented as spike trains. The spike trains determine when motor unit sources fire in time. Typically, the spikes are treated as binary (on/off), although more complex interpretations are also contemplated. The decomposed MUAPs can be used for various purposes, such as the intent decoding 34 and/or neuromuscular assessment 36 of FIG. 1. Additionally or alternatively, the decomposed MUAPs represented as spike trains in source space can be visualized 90. Based on the firing timings, waveforms can be extracted from the original EMG signal. In another approach, the motor unit sources can be visualized using a heatmap of the backward filter 52.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. An electromyography (EMG) measurement device comprising:
a garment configured to be worn on an anatomical region of an associated wearer;
a plurality of electrodes arranged on the garment to contact skin of the anatomical region when the garment is worn on the anatomical region of the associated wearer;
electronics operatively connected with the plurality of electrodes and configured to measure EMG data emanating from the anatomical region; and
an electronic processor programmed to filter the EMG data to suppress or remove artifacts using filters computed using approximate joint diagonalization of covariance (AJDC) matrices or by transforming the EMG data to source signals using iteratively adjusted forward filters.

2. The EMG measurement device of claim 1, wherein the electronic processor is programmed to filter the EMG data by operations including:
transforming the EMG data to source signals using forward filters computed using the AJDC matrices;
adjusting diagonal elements of a weighting matrix to suppress artifact sources and/or upscale non-artifact sources; and
transforming the source signals using backward filters and the weighting matrix with the adjusted diagonal elements to generate filtered EMG data having suppressed or removed artifacts.

3. The EMG measurement device of claim 2, wherein the adjusting of the diagonal elements of the weighting matrix includes setting diagonal elements of the weighting matrix corresponding to artifact sources to zero.

4. The EMG measurement device of claim 2, wherein the adjusting of the diagonal elements of the weighting matrix includes upscaling diagonal elements of the weighting matrix not corresponding to artifact sources.

5. The EMG measurement device of claim 2, wherein the adjusting of the diagonal elements of the weighting matrix further includes:
identifying artifact sources by applying one or more thresholds.

6. The EMG measurement device of claim 1, wherein the electronic processor is programmed to filter the EMG data to suppress or remove artifacts by transforming the EMG data to source signals using iteratively adjusted forward filters that are iteratively adjusted to identify motor unit action potentials (MUAPs) extracted from the source signals.

7. The EMG measurement device of claim 6, wherein the MUAPs are extracted by operations including:
computing power signals corresponding to the source signals by squaring the respective source signals; and
identifying the MUAPs from the computed power signals using peak detection.

8. The EMG measurement device of claim 7, wherein the identification of the MUAPs from the computed power signals using peak detection includes:
removing low pulse-to-noise ratio signals by clustering peak signals of the power signals.

9. The EMG measurement device of claim 6, further comprising:
a neuromuscular electrical stimulation (NMES) stimulator operatively connected with the electrodes;
wherein the electronic processor is further programmed to operate the NMES stimulator based on the identified MUAPs to deliver functional electrical stimulation to cause movement of the anatomical region.

10. The EMG measurement device of claim 6, wherein the electronic processor is further programmed to perform a neuromuscular debilitation assessment based on the identified MUAPs.

11. An electromyography (EMG) measurement method comprising:
measuring EMG data emanating from an anatomical region; and
filtering the EMG data to suppress or remove artifacts using filters computed using approximate joint diagonalization of covariance (AJDC) matrices or by transforming the EMG data to source signals using iteratively adjusted forward filters.

12. The EMG measurement method of claim 11, wherein the filtering includes:
transforming the EMG data to source signals using forward filters computed using AJDC matrices; adjusting diagonal elements of a weighting matrix to suppress artifact sources and/or upscale non-artifact sources; and
transforming the source signals using backward filters and the weighting matrix with the adjusted diagonal elements to generate filtered EMG data having suppressed or removed artifacts.

13. The EMG measurement method of claim 12, wherein the adjusting of the diagonal elements of the weighting matrix includes setting diagonal elements of the weighting matrix corresponding to artifact sources to zero.

14. The EMG measurement method of claim 12, wherein the adjusting of the diagonal elements of the weighting matrix includes upscaling diagonal elements of weighting matrix not corresponding to artifact sources.

15. The EMG measurement method of claim 12, wherein the adjusting of the diagonal elements of the weighting matrix further include:
identifying artifact sources in the EMG data in source space by applying one or more thresholds.

16. The EMG measurement method of claim 11, wherein the filtering includes:
filtering the EMG data to suppress or remove artifacts by transforming the EMG data to source signals using iteratively adjusted forward filters that are iteratively adjusted to identify motor unit action potentials (MUAPs) extracted from the signal sources.

17. The EMG measurement method of claim 16, wherein the MUAPs are extracted by operations including:
computing power signals corresponding to the source signals by squaring the respective source signals; and
identifying the MUAPs from the computed power signals using peak detection.

18. The EMG measurement method of claim 17, wherein the identification of the MUAPs from the computed power signals using peak detection includes:
removing low pulse-to-noise ratio signals by clustering peak signals of the power signals.

19. An electronic processor programmed to perform motor unit action potential (MUAP) extraction on electromyography (EMG) data by operations including:
transforming the EMG data to source signals using forward filters;
computing power signals corresponding to the source signals by squaring the respective source signals; and
identifying the MUAPs from the computed power signals using peak detection.

20. The electronic processor of claim 19, wherein the performing of the MUAP extraction on the EMG data further includes:
iteratively repeating the transforming, computing, and identifying to iteratively add to the identified MUAPs.
